(19) United States
(12) Patent Application Publication
(10) Pub. No.: US 2023/0310505 A1
(43) Pub. Date: Oct. 5, 2023

(54) COMPOSITION FOR PREVENTING HEARING LOSS CONTAINING MESENCHYMAL STEM CELLS OR EXOSOMES DERIVED THEREFROM AS ACTIVE INGREDIENT

(71) Applicant: UNIVERSITY INDUSTRY FOUNDATION, YONSEI UNIVERSITY WONJU CAMPUS, Gangwon-do (KR)

(72) Inventors: Young Joon SEO, Gangwon-do (KR); Dong Jun PARK, Gangwon-do (KR); Jeong Eun PARK, Gangwon-do (KR)

(21) Appl. No.: 18/006,684

(22) PCT Filed: Jun. 21, 2021
(86) PCT No.: PCT/KR2021/007719

§ 371 Date: Jan. 24, 2023

(30) Foreign Application Priority Data
Jul. 27, 2020 (KR) 10-2020-0092761
Jul. 27, 2020 (KR) 10-2020-0092762

(51) Int. Cl. A61K 35/28; C12N 5/0775; A61P 27/16

(52) U.S. Cl. CPC A61K 35/28; C12N 5/0665; A61P 27/16

(57) ABSTRACT

The present invention relates to a composition for preventing hearing loss, containing mesenchymal stem cells (MSC) or exosomes derived therefrom as an active ingredient. Specifically, an effect of preventing damage to the inner hair cells and outer hair cells of the cochlea caused by ototoxic drugs is exerted since a large amount of HSP70 protein is contained in exosomes contained in an MSC culture solution obtained according to the present invention, MSCs co-cultured with cochlear explants according to the present invention, and exosomes contained in a co-culture solution of MSCs and cochlear explants, and thus the MSCs co-cultured with cochlear explants, culture solution, and exosomes isolated therefrom can be usefully utilized as an active ingredient of a composition for preventing hearing loss.

### CROSS-REFERENCE TO RELATED APPLICATIONS

This application is a U.S. National Stage application of PCT/KR2021/007719 filed 21 Jun. 2021, which claims priority to Korean Patent Application No. 10-2020-0092761 filed 27 Jul. 2020, and Korean Patent Application No. 10-2020-0092762 filed 27 Jul. 2020, the entire disclosures of which are hereby incorporated by reference in their entireties.

### TECHNICAL FIELD

The present invention relates to a composition for preventing hearing loss, containing mesenchymal stem cells (MSC) or exosomes derived therefrom as an active ingredient, specifically to a pharmaceutical composition for preventing hearing loss, containing mesenchymal stem cells co-cultured with cochlear explants, a co-culture solution of mesenchymal stem cells and cochlear explants or exosomes isolated therefrom, or a mesenchymal stem cell culture solution or an exosomes isolated therefrom as an active ingredient.

### BACKGROUND ART

Deafness, namely hearing loss is divided into conductive hearing loss occurring when the outer ear and middle ear, which are organs that transmit sound, are infected with diseases such as inflammation and sensorineural hearing loss caused by problems in the cochlea, an organ that detects sound, the auditory nerve, which transmits sound with electrical energy, and the brain responsible for hearing, which plays a comprehensive role in discriminating and understanding sound, and is a common disease affecting about 15% to 20% of the population.

Sensorineural hearing loss may be caused by inflammatory disease such as labyrinthitis or meningitis, noise, ototoxic drugs, trauma such as temporal bone fracture, geriatric deafness, Meniere's disease, metabolic abnormalities such as hypothyroidism, brain ischemic disease, blood disease such as leukemia, neurological abnormality such as multiple sclerosis, immune abnormality, neoplastic disease such as acoustic nerve tumor, bone disease or the like.

Aminoglycoside antibiotics are one of the representative ototoxic drugs. Aminoglycoside antibiotics include streptomycin, kanamycin, gentamicin, neomycin, amikacin, tobramycin, netilmicin, dibekacin, sisomycin and the like, and are mainly used for gram-negative bacterial infections, tuberculosis, and deep infections that do not respond well to general antibiotics. Aminoglycoside antibiotics have side effects such as ototoxicity and renal toxicity that causes hearing and equilibrium dysfunction in the inner ear. The side effects may be caused by overdose as well as by long-term use at therapeutic doses, and in some cases, ototoxicity is caused even by short-term use at appropriate doses. As ototoxicity caused by aminoglycoside antibiotics, approximately 15% of users have vestibular dysfunction and 10% to 30% of users have hearing loss, and ototoxicity mainly occurs in both ears in the form of sudden severe hearing loss at high frequencies of 4000 Hz or higher. In particular, about 4 million patients in the United States are being treated with aminoglycoside antibiotics, and among these, up to 10% of patients receiving intravenous administration of the antibiotics suffer from hearing loss caused by aminoglycosides.

Cisplatin, an anticancer drug, is also one of the representative ototoxic drugs, causes serious side effects such as hearing and equilibrium dysfunction, and causes irreversible bilateral sensorineural hearing loss. It has been reported that such ototoxicity of cisplatin anticancer drug causes hearing impairment in about 30% of cisplatin users, and, the incidence of hearing impairment is higher in children with about 50% of child users being affected. However, cisplatin is still widely used because of its excellent anticancer treatment effect.

This hearing loss may be temporary, but occurs irreversibly in most patients. It is difficult to predict the onset of hearing loss in the early stages, and significant hearing loss may occur even after a single administration of antibiotics. Since hearing loss may occur weeks or months after completion of antibiotic or anticancer treatment, drug-induced ototoxicity is determined after hearing loss has occurred in drug-administered patients. Therefore, it is necessary to prepare potential treatment alternatives before the onset of hearing loss in patients.

Meanwhile, mesenchymal stem cells are cells of stromal origin, have the characteristics of self-renewal, can differentiate into bone, cartilage, adipose tissue, muscle, tendon, ligament, nerve tissue and the like, and thus are attracting attention as cells suitable for cell therapy. Mesenchymal stem cells have been reported to be useful for regenerating damaged tissues by osteogenesis imperfecta, myocardial infarction, lung injury, brain infarction and the like. Currently, it is attempted in several studies to utilize the differentiation potency and regenerative ability of mesenchymal stem cells for use as therapeutic agents.

Accordingly, the present inventors have made efforts to develop a substance capable of preventing hearing loss from occurring before the onset of hearing loss, and as a result, confirmed that HSP70 protein and exosomes containing the protein increase in mesenchymal stem cells co-cultured with cochlear explants according to the present invention and that this is effective in preventing damage to inner hair cells and outer hair cells of the cochlea caused by ototoxic drugs. It has also been confirmed that exosomes containing HSP70 protein increase in a culture solution obtained by co-culturing mesenchymal stem cells and cochlear explants according to the present invention. In addition, it has been confirmed that an effect of preventing damage to inner hair cells and outer hair cells of the cochlea caused by ototoxic drugs is exerted since HSP70 protein is contained in exosomes isolated from mesenchymal stem cells, whereby the present invention has been completed.

### CITATION LIST

### Patent Literature

### Patent Literature 1

Korean Patent Publication No. 10-2013-0012552

### Non Patent Literature

### Non Patent Literature 1

Doreen Rosenstrauch, et al. Stem cell therapy for ischemic heart failure. Tex Heart Inst J. 2005; 32(3): 339-347.

### Non Patent Literature 2

Rojas M, et al. Bone Marrow-Derived Mesenchymal Stem Cells in Repair of the Injured Lung, Am J Respir Cell Mol Biol. 2005; 33:145-52.

### Non Patent Literature 3

Y Takada et al. Ototoxicity-induced loss of hearing and inner hair cells is attenuated by HSP70 gene transfer. Methods & Clinical Development. 2015; 2:15019.

### SUMMARY OF INVENTION

### Technical Problem

An object of the present invention is to provide a pharmaceutical composition for preventing hearing loss, containing mesenchymal stem cells (MSC) co-cultured with cochlear explants, a co-culture solution of mesenchymal stem cells and cochlear explants or exosomes isolated therefrom as an active ingredient; a method for preventing hearing loss, which comprises administering a pharmaceutically effective amount of mesenchymal stem cells co-cultured with cochlear explants, a co-culture solution of mesenchymal stem cells and cochlear explants or exosomes isolated therefrom to a subject; and a use of mesenchymal stem cells co-cultured with cochlear explants, a co-culture solution of mesenchymal stem cells and cochlear explants or exosomes isolated therefrom for preparation of a pharmaceutical composition for preventing hearing loss.

Another object of the present invention is to provide a pharmaceutical composition for preventing hearing loss, containing mesenchymal stem cell-derived exosomes as an active ingredient; a method for preventing hearing loss, which comprises administering a pharmaceutically effective amount of a mesenchymal stem cell culture solution or exosomes isolated therefrom to a subject; and a use of a mesenchymal stem cell culture solution or exosomes isolated therefrom for preparation of a pharmaceutical composition for preventing hearing loss.

### Solution to Problem

In order to achieve the objects, the present invention provides a pharmaceutical composition for preventing hearing loss, containing mesenchymal stem cells (MSC) co-cultured with cochlear explants, a co-culture solution of mesenchymal stem cells and cochlear explants or exosomes isolated therefrom as an active ingredient; a method for preventing hearing loss, which comprises administering a pharmaceutically effective amount of mesenchymal stem cells co-cultured with cochlear explants, a co-culture solution of mesenchymal stem cells and cochlear explants or exosomes isolated therefrom to a subject; and a use of mesenchymal stem cells co-cultured with cochlear explants, a co-culture solution of mesenchymal stem cells and cochlear explants or exosomes isolated therefrom for preparation of a pharmaceutical composition for preventing hearing loss.

The present invention also provides the pharmaceutical composition for preventing hearing loss, further containing cochlear explants co-cultured with mesenchymal stem cells; the method for preventing hearing loss, which comprises further administering cochlear explants co-cultured with mesenchymal stem cells; and the use of cochlear explants co-cultured with mesenchymal stem cells for preparation of a pharmaceutical composition for preventing hearing loss.

The present invention also provides a pharmaceutical composition for preventing hearing loss, containing a mesenchymal stem cell culture solution or exosomes isolated therefrom as an active ingredient; a method for preventing hearing loss, which comprises administering a pharmaceutically effective amount of a mesenchymal stem cell culture solution or exosomes isolated therefrom to a subject; and the use of a mesenchymal stem cell culture solution or exosomes isolated therefrom for preparation of a pharmaceutical composition for preventing hearing loss.

The present invention also provides a method for preparing mesenchymal stem cells for preventing hearing loss, which comprises:
1) co-culturing cochlear explants and mesenchymal stem cells; and
2) obtaining co-cultured mesenchymal stem cells. The present invention also provides a method for preparing mesenchymal stem cells and cochlear explants for preventing hearing loss, which comprises co-culturing cochlear explants and mesenchymal stem cells.

The present invention also provides a method for preparing a co-culture solution of mesenchymal stem cells and cochlear explants for preventing hearing loss, which comprises:
1) co-culturing mesenchymal stem cells and cochlear explants in a co-culture medium; and
2) recovering a cell culture supernatant of co-cultured mesenchymal stem cells and cochlear explants.

The present invention also provides a method for preparing exosomes isolated from a co-culture solution of mesenchymal stem cells and cochlear explants for preventing hearing loss, which comprises:
1) co-culturing mesenchymal stem cells and cochlear explants in a co-culture medium; and
2) recovering a cell culture supernatant of co-cultured mesenchymal stem cells and cochlear explants; and
3) isolating exosomes from the recovered cell culture supernatant and purifying the exosomes.

### Advantageous Effects of Invention

An effect of preventing damage to the inner hair cells and outer hair cells of the cochlea caused by ototoxic drugs is exerted since HSP70 protein and exosomes containing the protein increase in mesenchymal stem cells co-cultured with cochlear explants according to the present invention and a large amount of HSP70 protein is contained in exosomes contained in a co-culture solution of mesenchymal stem cells and cochlear explants, and thus mesenchymal stem cells co-cultured with cochlear explants according to the present invention, a co-culture solution of mesenchymal stem cells and cochlear explants, and exosomes isolated from the co-culture solution can be usefully utilized as an active ingredient of a composition for preventing hearing loss.

An effect of preventing damage to the inner hair cells and outer hair cells of the cochlea caused by ototoxic drugs is exerted since a large amount of HSP70 protein is contained in exosomes contained in a mesenchymal stem cell culture solution, and thus a mesenchymal stem cell culture solution and exosomes isolated therefrom can be usefully utilized as an active ingredient of a composition for preventing hearing loss.

### BRIEF DESCRIPTION OF DRAWINGS

FIGS. 1A to 1C are diagrams confirming the cell viability of inner hair cells (IHC) and outer hair cells (OHC) after treatment of cochlear explants of basal turn, middle turn, and apical turn with cisplatin:

FIG. 1A is a diagram schematically illustrating a method for treating cochlear explants with cisplatin;

FIG. 1B is a diagram confirming the cell viability of IHCs and OHCs in cochlear explants of basal turn, middle turn, and apical turn after treatment with cisplatin by immunofluorescence analysis; and

FIG. 1C is a graph illustrating the cell viability of IHCs and OHCs in cochlear explants of basal turn, middle turn, and apical turn after treatment with cisplatin.

FIGS. 2A to 2C are diagrams confirming mesenchymal stem cells (MSC) isolated from human bone marrow and extracellular vesicular (EV) derived therefrom:

FIG. 2A is a diagram confirming MSCs isolated from human bone marrow using a CD marker;

FIG. 2B is a diagram confirming exosomes in MSCs isolated from human bone marrow and EVs derived therefrom; and

FIG. 2C is a diagram confirming the size and concentration of exosomes in EVs derived from MSCs isolated from human bone marrow.

FIGS. 3A to 3D are diagrams confirming the cell viability of IHCs and OHCs after co-culture of cochlear explants of basal turn, middle turn, and apical turn and MSCs isolated from human bone marrow and treatment with cisplatin:

FIG. 3A is a diagram schematically illustrating a method for co-culturing cochlear explants and MSCs isolated from human bone marrow;

FIG. 3B is a diagram illustrating groups having different time points of co-culture of cochlear explants and MSCs isolated from human bone marrow and different time points of treatment with cisplatin;

FIG. 3C is a diagram confirming the cell viability of IHCs and OHCs in cochlear explants of basal turn, middle turn, and apical turn after co-culture of cochlear explants and MSCs isolated from human bone marrow and treatment with cisplatin by immunofluorescence analysis; and

FIG. 3D is a graph illustrating the cell viability of IHCs and OHCs in cochlear explants of basal turn, middle turn, and apical turn after co-culture of cochlear explants and MSCs isolated from human bone marrow and treatment with cisplatin.

FIGS. 4A and 4B are diagrams confirming the cell viability of IHCs and OHCs in cochlear explants of basal turn, middle turn, and apical turn after co-culture of cochlear explants and MSCs isolated from human bone marrow for 2, 12, 18, 24 or 48 hours, removal of MSCs, and treatment with cisplatin:

FIG. 4A is a diagram confirming the cell viability of IHCs and OHCs in cochlear explants of basal turn, middle turn, and apical turn after co-culture of cochlear explants and MSCs isolated from human bone marrow for 2, 12, 18, 24 or 48 hours, removal of MSCs, and treatment with cisplatin by immunofluorescence analysis; and

FIG. 4B is a graph illustrating the cell viability of IHCs and OHCs in cochlear explants of basal turn, middle turn, and apical turn after co-culture of cochlear explants and MSCs isolated from human bone marrow for 2, 12, 18, 24 or 48 hours, removal of MSCs, and treatment with cisplatin.

FIGS. 5A to 5C are diagrams confirming the cell viability of IHCs and OHCs after treatment of cochlear explants of basal turn, middle turn, and apical turn with exosomes derived from MSCs isolated from human bone marrow and treatment with cisplatin:

FIG. 5A is a diagram schematically illustrating a method for treating cochlear explants with exosomes derived from MSCs isolated from human bone marrow;

FIG. 5B is a diagram confirming the cell viability of IHCs and OHCs after treatment of cochlear explants of basal turn, middle turn, and apical turn with exosomes derived from MSCs isolated from human bone marrow and treatment with cisplatin by immunofluorescence analysis: and

FIG. 5C is a graph illustrating the cell viability of IHCs and OHCs after treatment of cochlear explants of basal turn, middle turn, and apical turn with exosomes derived from MSCs isolated from human bone marrow and treatment with cisplatin.

FIGS. 6A to 6C are diagrams confirming the expression of exosome markers and HSP70 protein in cochlear explants, MSCs, and culture solutions thereof after culture of cochlear explants alone, culture of MSCs isolated from human bone marrow alone, or co-culture of cochlear explants and MSCs isolated from human bone marrow:

FIG. 6A is a diagram schematically illustrating a method for obtaining cochlear explants, MSCs, and culture solutions thereof after culture of cochlear explants alone, culture of MSCs isolated from human bone marrow alone, or co-culture of cochlear explants and MSCs isolated from human bone marrow;

FIG. 6B is a diagram confirming the expression of exosome markers and HSP70 protein in culture solutions of cochlear explants and MSCs after culture of cochlear explants alone, culture of MSCs isolated from human bone marrow alone, or co-culture of cochlear explants and MSCs isolated from human bone marrow; and

FIG. 6C is a diagram confirming the expression of exosome markers and HSP70 protein in cochlear explants and MSCs after culture of cochlear explants alone, culture of MSCs isolated from human bone marrow alone, or co-culture of cochlear explants and MSCs isolated from human bone marrow.

### DESCRIPTION OF EMBODIMENTS

Hereinafter, the present invention will be described in more detail.

The present invention provides a pharmaceutical composition for preventing hearing loss, containing mesenchymal stem cells (MSC) co-cultured with cochlear explants, a co-culture solution of mesenchymal stem cells and cochlear explants or exosomes isolated therefrom as an active ingredient; a method for preventing hearing loss, which comprises administering a pharmaceutically effective amount of mesenchymal stem cells co-cultured with cochlear explants, a co-culture solution of mesenchymal stem cells and cochlear explants or exosomes isolated therefrom to a subject; and a use of mesenchymal stem cells co-cultured with cochlear explants, a co-culture solution of mesenchymal stem cells and cochlear explants or exosomes isolated therefrom for preparation of a pharmaceutical composition for preventing hearing loss.

In the present invention, the mesenchymal stem cells and the cochlear explants may be co-cultured in a state of being spatially separated from each other in a co-culture medium. For example, culture may be performed in a state where the mesenchymal stem cells share the same medium with the cochlear explants in the same culture vessel or the mesenchymal stem cells and the cochlear explants do not come into physical contact with each other in the respective culture media. Specifically, the co-culture may be performed using a transwell. More specifically, inoculation may be performed so that the mesenchymal stem cells are positioned in the transwell upper chamber and the cochlear explants are positioned in the transwell lower chamber, and the mesenchymal stem cells and the cochlear explants may be co-cultured in a co-culture medium, for example, while sharing the same medium or in the respective culture media while being spatially separated from each other. When cultured this way, by the interaction between the mesenchymal stem cells and the cochlear explants, the mesenchymal stem cells may secrete various substances that may affect the cochlear explants, and specifically, factors capable of inhibiting damage to auditory hair cells, for example, HSP70 protein and exosomes containing the same, may increase in the mesenchymal stem cells.

The “co-culture solution of mesenchymal stem cells and cochlear explants” refers to a culture supernatant obtained by co-culturing the mesenchymal stem cells and the cochlear explants in a co-culture medium. Specifically, the co-culture solution of mesenchymal stem cells and cochlear explants may be a culture supernatant obtained by co-culturing the mesenchymal stem cells and the cochlear explants in a state of being spatially separated from each other in a co-culture medium, more specifically a culture supernatant obtained by co-culturing the mesenchymal stem cells and the cochlear explants in a co-culture medium, for example, while sharing the same medium or in the respective culture media while being spatially separated from each other as the mesenchymal stem cells are positioned in the transwell upper chamber and the cochlear explants are positioned in the transwell lower chamber. When cultured this way, by the interaction between the mesenchymal stem cells and the cochlear explants, the mesenchymal stem cells may secrete various substances that may affect the cochlear explants into the co-culture medium, and thus factors capable of inhibiting damage to auditory hair cells, for example, HSP70 protein and exosomes containing the same, may increase in the mesenchymal stem cells in the co-culture medium.

In the present invention, the mesenchymal stem cells and cochlear explants may be separated by methods known in the art and can be grown in conventional media. The medium contains nutrients required by the cells to be cultured, that is, cultured cells, in order to culture specific cells, and substances for special purposes may be additionally added to and mixed in the medium. The medium is also referred to as an incubator or a culture solution, and is a concept that includes all of a natural medium, a synthetic medium, or a selective medium. For example, as the medium for the mesenchymal stem cells, any medium for culturing mesenchymal stem cells may be used without limitation, for example, a low-glucose DMEM medium may be used as a commercially available medium, but the medium is not limited thereto. As the medium for the cochlear explants, any medium for culturing cochlear explants may be used without limitation, for example, DMEM/F12 medium may be used as a commercially available medium, but the medium is not limited thereto. Accordingly, the co-culture medium may be a medium for culturing mesenchymal stem cells and/or a medium for culturing cochlear explants, but is not limited thereto.

The mesenchymal stem cells and cochlear explants may be co-cultured according to conventional culture methods. For example, the mesenchymal stem cells may be inoculated at a cell number of 1×10³ to 1×10¹⁰, specifically a cell number of 1×10⁴ to 1×10⁷, and the co-culture may be performed at a temperature of 35° C. to 40° C., preferably 36° C. to 38° C. and 4% to 6% CO₂, but is not limited thereto.

The co-culture may be performed for 12 hours or more, specifically 18 hours or more, more specifically 18 to 48 hours, but is not limited thereto. However, in the case of co-culture for less than 12 hours, secretion of factors capable of inhibiting damage to auditory hair cells, for example, HSP70 protein and exosomes containing the same, may be insufficient.

In the present invention, the mesenchymal stem cells include mesenchymal stem cells derived from all mammals such as humans, monkeys, pigs, horses, cows, sheep, dogs, cats, mice, and rabbits, but may specifically be mesenchymal stem cells derived from humans.

The mesenchymal stem cells may be mesenchymal stem cells derived from bone marrow, fat, umbilical cord, umbilical cord blood or tonsil, specifically mesenchymal stem cells derived from bone marrow, but are not limited thereto.

In the present invention, as the mesenchymal stem cells and cochlear explants are co-cultured, the expression of HSP70 increases and the secretion of exosomes in which expression of HSP70 is increased increases. Accordingly, the mesenchymal stem cells can protect inner hair cells and outer hair cells of the cochlea from damage.

In the present invention, the exosomes are specifically a membrane-structured vesicle secreted from mesenchymal stem cells and/or cochlear explants, are CD63 positive, and contain HSP70 protein to protect inner hair cells and outer hair cells of the cochlea from damage. In particular, the expression of HSP70 in the above-mentioned exosomes is increased by the interactions between mesenchymal stem cells and cochlear explants than that in the exosomes secreted from each of mesenchymal stem cells and cochlear explants, and the effect of protecting inner hair cells and outer hair cells of the cochlea from damage is further enhanced.

The exosomes may have a diameter of 40 to 180 nm, specifically a diameter of 50 to 150 nm, more specifically a diameter of 60 to 100 nm, but are not limited thereto, and the diameter of exosomes may vary depending on the cell type to be isolated, the isolation method, and the measurement method.

In the present invention, the hearing loss may be sensorineural hearing loss, and the sensorineural hearing loss may be specifically ototoxic hearing loss, organ of Corti damage-induced hearing loss due to viral infection, chronic otitis media-induced hearing loss, age-related hearing loss, noise-induced hearing loss, sudden hearing loss, autoimmune hearing loss, vascular ischemic hearing loss, head injury hearing loss, or hereditary hearing loss, more specifically ototoxic hearing loss, but is not limited thereto.

The ototoxic hearing loss is caused by an ototoxic drug, and the ototoxic drug may be specifically cisplatin, carboplatin, amikacin, arbekacin, kanamycin, gentamicin, neomycin, netilmicin, dibekacin, sisomycin, streptomycin, tobramycin, livodomycin, paromomycin, acetazolamide, furosemide, bumetanide or ethacrynic acid, more specifically cisplatin, but is not limited thereto.

The sensorineural hearing loss may be caused by damage to inner hair cells of the cochlea, outer hair cells of the cochlea or surrounding tissues.

In the present invention, the composition for preventing hearing loss may further contain co-cultured cochlear explants together with mesenchymal stem cells co-cultured with the cochlear explants.

The cochlear explants are cochlear explants used for co-culture of mesenchymal stem cells, and by the interaction between the mesenchymal stem cells and the cochlear explants, factors capable of inhibiting damage to auditory hair cells, for example, HSP70 protein and exosomes containing the same, may increase in the cochlear explants as well as the mesenchymal stem cells.

In a specific embodiment of the present invention, the present inventors have obtained cochlear explants and human bone marrow-derived mesenchymal stem cells.

The present inventors have confirmed that mesenchymal stem cells co-cultured with cochlear explants are effective in preventing damage to auditory hair cells caused by ototoxic drugs as the viability of hair cells of cochlear explants is excellent when the cochlear explants and mesenchymal stem cells are co-cultured in a state of being spatially separated from each other using a transwell, the mesenchymal stem cells are then removed, and treatment with cisplatin is performed in that state, that is, when pretreatment of cochlear explants with mesenchymal stem cells is performed and then treatment with cisplatin is performed.

The present inventors have confirmed that the mesenchymal stem cells co-cultured with cochlear explants for 18 hours or more are more effective in preventing damage to hair cells of the cochlear explants caused by cisplatin.

The present inventors have confirmed that HSP70 protein and exosomes containing the protein increase in the mesenchymal stem cells co-cultured with cochlear explants.

The present inventors have also confirmed that HSP70 protein and exosomes containing the protein increase in cochlear explants co-cultured with mesenchymal stem cells as well as in mesenchymal stem cells co-cultured with cochlear explants.

Accordingly, the present inventors have confirmed that HSP70 protein and exosomes containing the protein increase in mesenchymal stem cells co-cultured with cochlear explants and that this is effective in preventing damage to auditory hair cells caused by ototoxic drugs, and thus mesenchymal stem cells co-cultured with cochlear explants according to the present invention can be usefully utilized as an active ingredient of a composition for preventing hearing loss.

The present inventors have confirmed that HSP70 protein and exosomes containing the protein increase in cochlear explants used for co-culture of mesenchymal stem cells as well and this can prevent damage to auditory hair cells caused by ototoxic drugs, and thus cochlear explants together with mesenchymal stem cells co-cultured with the cochlear explants according to the present invention can be usefully utilized as an active ingredient of a composition for preventing hearing loss.

In a specific embodiment of the present invention, the present inventors have confirmed that mesenchymal stem cells co-cultured with cochlear explants are effective in preventing damage to auditory hair cells caused by ototoxic drugs as the viability of hair cells of cochlear explants is excellent when the obtained cochlear explants and human bone marrow-derived mesenchymal stem cells are co-cultured in a state of being spatially separated from each other using a transwell, the mesenchymal stem cells are then removed, and treatment with cisplatin is performed in that state, that is, when pretreatment of cochlear explants with mesenchymal stem cells is performed and then treatment with cisplatin is performed.

The present inventors have confirmed that the mesenchymal stem cells co-cultured with cochlear explants for 18 hours or more are more effective in preventing damage to hair cells of the cochlear explants caused by cisplatin.

The present inventors have confirmed that the expression of exosome marker CD63 and HSP70 protein is higher in a co-culture solution of mesenchymal stem cells and cochlear explants than in a culture solution of cochlear explants.

Accordingly, the present inventors have confirmed that exosomes containing HSP70 increase in a co-culture solution of mesenchymal stem cells and cochlear explants and this further enhances the effect of preventing damage to auditory hair cells caused by ototoxic drugs, and thus a co-culture solution of mesenchymal stem cells and cochlear explants and exosomes isolated therefrom can be usefully utilized as an active ingredient of a composition for preventing hearing loss.

The present invention also provides a pharmaceutical composition for preventing hearing loss, containing a mesenchymal stem cell culture solution or exosomes isolated therefrom as an active ingredient; a method for preventing hearing loss, which comprises administering a pharmaceutically effective amount of a mesenchymal stem cell culture solution or exosomes isolated therefrom to a subject; and a use of a mesenchymal stem cell culture solution or exosomes isolated therefrom for preparation of a pharmaceutical composition for preventing hearing loss.

In the present invention, the “mesenchymal stem cell culture solution” refers to a cell culture supernatant obtained by culturing mesenchymal stem cells in a culture medium. The mesenchymal stem cell culture solution contains various physiologically active substances secreted from the cells during the mesenchymal stem cell culturing process. For example, in the process of culturing mesenchymal stem cells, exosomes containing HSP70 are secreted from the cells to protect inner hair cells and outer hair cells of the cochlea from damage.

The mesenchymal stem cells include mesenchymal stem cells derived from all mammals such as humans, monkeys, pigs, horses, cows, sheep, dogs, cats, mice, and rabbits, but may specifically be mesenchymal stem cells derived from humans.

The mesenchymal stem cells may be mesenchymal stem cells derived from bone marrow, fat, umbilical cord, umbilical cord blood or tonsil, specifically mesenchymal stem cells derived from bone marrow, but are not limited thereto.

The mesenchymal stem cells may be cultured in a conventional culture medium according to a conventional culture method. The medium contains nutrients required by the cells to be cultured, that is, cultured cells, in order to culture specific cells, and substances for special purposes may be additionally added to and mixed in the medium. The medium is also referred to as an incubator or a culture solution, and is a concept that includes all of a natural medium, a synthetic medium, or a selective medium. For example, as the medium for the mesenchymal stem cells, any medium for culturing mesenchymal stem cells may be used without limitation, for example, a low-glucose DMEM medium may be used as a commercially available medium, but the medium is not limited thereto. For example, the mesenchymal stem cells may be inoculated at a cell number of 1×10³ to 1×10¹⁰, specifically a cell number of 1×10⁴ to 1×10⁷, and the co-culture may be performed at a temperature of 35° C. to 40° C., preferably 36° C. to 38° C. and 4% to 6% CO₂, but is not limited thereto.

In the present invention, the exosomes are a membrane-structured vesicle secreted from cells, are known to play various roles such as delivering membrane components, proteins, nucleic acids and the like by binding to other cells and tissues, and mean to include both vesicles (for example, exosome-like vesicles) having a composition similar to that of exosomes and microvesicles. Specifically, the exosomes are a membrane-structured vesicle secreted from mesenchymal stem cells, are CD63 positive, and contain HSP70 protein to protect inner hair cells and outer hair cells of the cochlea from damage.

The exosomes may have a diameter of 40 to 180 nm, specifically a diameter of 50 to 150 nm, more specifically a diameter of 60 to 100 nm, but are not limited thereto, and the diameter of exosomes may vary depending on the cell type to be isolated, the isolation method, and the measurement method.

The exosomes may be prepared using an exosome isolating method known in the art, and the preparation may be performed, for example, according to the following steps, but is not limited thereto:
1) culturing mesenchymal stem cells in a culture medium;
2) recovering the cell culture supernatant;
3) centrifuging the recovered cell culture supernatant; and
4) isolating and purifying exosomes.

In the present invention, the hearing loss may be sensorineural hearing loss, and the sensorineural hearing loss may be specifically ototoxic hearing loss, organ of Corti damage-induced hearing loss due to viral infection, chronic otitis media-induced hearing loss, age-related hearing loss, noise-induced hearing loss, sudden hearing loss, autoimmune hearing loss, vascular ischemic hearing loss, head injury hearing loss, or hereditary hearing loss, more specifically ototoxic hearing loss, but is not limited thereto.

The ototoxic hearing loss is caused by an ototoxic drug, and the ototoxic drug may be specifically cisplatin, carboplatin, amikacin, arbekacin, kanamycin, gentamicin, neomycin, netilmicin, dibekacin, sisomycin, streptomycin, tobramycin, livodomycin, paromomycin, acetazolamide, furosemide, bumetanide or ethacrynic acid, more specifically cisplatin, but is not limited thereto.

The sensorineural hearing loss may be caused by damage to inner hair cells of the cochlea, outer hair cells of the cochlea or surrounding tissues.

In a specific embodiment of the present invention, the present inventors have obtained cochlear explants and human bone marrow-derived mesenchymal stem cells.

The present inventors have cultured the mesenchymal stem cells and observed extracellular vesicles (EV) in the culture solution, and have confirmed that most of the EVs are exosomes smaller than 100 nm.

When the culture solution is centrifuged to obtain a pellet containing exosomes and cochlear explants are treated with this pellet and then with cisplatin, it has been confirmed that the viability of hair cells of cochlear explants is excellent, and it has been confirmed that the expression of exosome marker CD63 and HSP70 protein is higher in the culture solution than in the cochlear explants.

Accordingly, the present inventors have confirmed that exosomes containing HSP70 is contained in a mesenchymal stem cell culture solution and that this is effective in preventing damage to auditory hair cells caused by ototoxic drugs, and thus a mesenchymal stem cell culture solution and exosomes isolated therefrom can be usefully utilized as an active ingredient of a composition for preventing hearing loss.

The pharmaceutical composition according to the present invention may contain mesenchymal stem cells in a dosage of 1.0×10³ to 1.0×10⁸ cells/kg (body weight). The pharmaceutical composition according to the present invention may contain a mesenchymal stem cell culture solution or exosomes isolated therefrom as an active ingredient, and may contain a co-culture solution of mesenchymal stem cells and cochlear explants or exosomes isolated therefrom as an active ingredient. However, the dosage may be prescribed in various ways depending on factors such as formulation method, administration method, and age, weight, sex, morbid condition, food, administration time, administration route, excretion rate and response sensitivity of the patient, and those skilled in the art can appropriately adjust the dosage in consideration of these factors. The number of administrations can be one time or two or more times within the range of clinically acceptable side effects, and the administration site may be one or two or more sites. The pharmaceutical composition may be administered to non-human animals in the same dosage per kg as that for humans, or in an amount acquired by converting the above-mentioned dosage to, for example, a volume ratio (for example, average value) of an organ (heart or the like) between a target animal and a human. Possible routes of administration include oral, sublingual, parenteral (for example, subcutaneous, intramuscular, intraarterial, intraperitoneal, intrathecal, or intravenous), rectal, and topical (including transdermal) administration, inhalation, and injection, or implantation of implantable devices or substances. Examples of animals to be treated include humans and other target mammals, and specifically include humans, monkeys, mice, rats, rabbits, sheep, cows, dogs, horses, and pigs.

The pharmaceutical composition according to the present invention may contain pharmaceutically acceptable carriers and/or additives. The pharmaceutical composition may contain, for example, sterile water, physiological saline, conventional buffers (phosphoric acid, citric acid, other organic acids, and the like), stabilizers, salts, antioxidants (ascorbic acid and the like), surfactants, suspending agents, tonicity agents, or preservatives. For topical administration, the pharmaceutical composition may contain organic substances such as biopolymers and inorganic substances such as hydroxyapatite, and specifically, a collagen matrix, a polylactic acid polymer or copolymer, a polyethylene glycol polymer or copolymer, and a combination of chemical derivatives thereof. In a case where the pharmaceutical composition according to an embodiment is prepared into a dosage form suitable for injection, mesenchymal stem cells and/or cochlear explants may be dissolved in a pharmaceutically acceptable carrier or frozen in a dissolved solution state.

The pharmaceutical composition according to the present invention may appropriately contain a suspending agent, a solubilizing agent, a stabilizer, an isotonic agent, a preservative, an adsorption inhibitor, a surfactant, a diluent, an excipient, a pH adjuster, a pain reliever, a buffer, a reducing agent, an antioxidant, and the like, if necessary depending on the administration method or dosage form. Pharmaceutically acceptable carriers and agents suitable for the present invention, including those exemplified above, are described in detail in the literature [Remington's Pharmaceutical Sciences, 19th ed., 1995]. The pharmaceutical composition according to the present invention may be prepared in a unit dose form or by being packaged in a multi-dose container through formulation using pharmaceutically acceptable carriers and/or excipients according to methods that can be easily performed by those skilled in the art to which the invention belongs. At this time, the dosage form may be in the form of a solution, suspension or emulsion in an oil or aqueous medium, or in the form of a powder, granule, tablet or capsule.

The present invention also provides a method for preparing mesenchymal stem cells for preventing hearing loss, which comprises:
1) co-culturing cochlear explants and mesenchymal stem cells; and
2) obtaining co-cultured mesenchymal stem cells.

In the method of the present invention, in step 1), the mesenchymal stem cells may be positioned in the transwell upper chamber and the cochlear explants may be positioned in the transwell lower chamber, and the mesenchymal stem cells and the cochlear explants may be co-cultured in a co-culture medium, specifically while sharing the same medium or in the respective culture media while being spatially separated from each other.

In step 1), the co-culture may be performed for 12 hours or more, specifically 18 hours or more, more specifically 18 to 48 hours, but is not limited thereto.

In the method of the present invention, it is easy to obtain only mesenchymal stem cells in step 2) since the co-cultured mesenchymal stem cells are positioned in the transwell upper chamber.

The cochlear explants, mesenchymal stem cells, culture method, and hearing loss are as described in the composition for preventing hearing loss containing mesenchymal stem cells co-cultured with cochlear explants as an active ingredient.

The present inventors have confirmed that HSP70 protein and exosomes containing the protein increase in mesenchymal stem cells co-cultured with cochlear explants in a state where the cochlear explants and the mesenchymal stem cells are spatially separated from each other using a transwell and that this is effective in preventing damage to auditory hair cells caused by ototoxic drugs, and thus the method for preparing mesenchymal stem cells according to the present invention can be usefully utilized as a method for preparing mesenchymal stem cells for preventing hearing loss.

The present invention also provides a method for preparing mesenchymal stem cells and cochlear explants for preventing hearing loss, which comprises co-culturing cochlear explants and mesenchymal stem cells.

In the method of the present invention, the cochlear explants, mesenchymal stem cells, culture method, and hearing loss are as described in the composition for preventing hearing loss containing mesenchymal stem cells co-cultured with cochlear explants as an active ingredient.

The present inventors have confirmed that HSP70 protein and exosomes containing the protein increase in mesenchymal stem cells co-cultured with cochlear explants in a state where the cochlear explants and the mesenchymal stem cells are spatially separated from each other using a transwell and in the cochlear explants used for co-culture of the mesenchymal stem cells and that this is effective in preventing damage to auditory hair cells caused by ototoxic drugs, and thus the method for preparing mesenchymal stem cells and cochlear explants according to the present invention can be usefully utilized as a method for preparing mesenchymal stem cells and cochlear explants for preventing hearing loss.

The present invention also provides a method for preparing a co-culture solution of mesenchymal stem cells and cochlear explants for preventing hearing loss, which comprises:
1) co-culturing mesenchymal stem cells and cochlear explants in a co-culture medium; and
2) recovering a cell culture supernatant of co-cultured mesenchymal stem cells and cochlear explants.

The present invention also provides a method for preparing exosomes isolated from a co-culture solution of mesenchymal stem cells and cochlear explants for preventing hearing loss, which comprises:
1) co-culturing mesenchymal stem cells and cochlear explants in a co-culture medium; and
2) recovering a cell culture supernatant of co-cultured mesenchymal stem cells and cochlear explants; and
3) isolating exosomes from the recovered cell culture supernatant and purifying the exosomes.

In the method of the present invention, the mesenchymal stem cells, cochlear explants, co-culture method, and hearing loss are as described in the composition for preventing hearing loss, so specific descriptions refer to the above contents, and only the configuration unique to the preparation method will be described below.

In the method of the present invention, in step 1), the mesenchymal stem cells and the cochlear explants may be co-cultured in a co-culture medium, for example, while sharing the same medium or in the respective culture media while being spatially separated from each other, more specifically, the mesenchymal stem cells and the cochlear explants may be co-cultured in a state of being spatially separated from each other in a co-culture medium as the mesenchymal stem cells are positioned in the transwell upper chamber and the cochlear explants are positioned in the transwell lower chamber.

In step 1), the co-culture may be performed for 12 hours or more, specifically 18 hours or more, more specifically 18 to 48 hours, but is not limited thereto.

The present inventors have confirmed that HSP70 protein and exosomes containing the same increase in a culture solution obtained by co-culturing mesenchymal stem cells and cochlear explants and that this is effective in preventing damage to inner hair cells and outer hair cells of the cochlea caused by ototoxic drugs, and thus the method for preparing a co-culture solution and exosomes isolated therefrom according to the present invention can be usefully utilized as a method for preparing a co-culture solution and exosomes isolated therefrom for preventing hearing loss.

### EXAMPLES

Hereinafter, the present invention will be described in detail by way of Examples and Experimental Examples.

However, the following Examples and Experimental Examples are merely illustrative of the present invention, and the contents of the present invention are not limited to the following Examples and Experimental Examples.

### Example 1 Isolation of Mesenchymal Stem Cells (MSC) from Human Bone Marrow

Human bone marrow was obtained from the iliac crest of patients who underwent transplantation treatment at Severance Christian Hospital in WonJu after obtaining written informed consent. Aspirates were collected with Vacutainers K2 EDTA (BD Biosciences). Mononuclear cells were diluted with PBS at 1:5 and isolated by density gradient centrifugation at 435×g for 20 minutes at room temperature using Ficoll hypaque solution (Gibco BRL, USA). The cell fraction was collected and cultured using DMEM-low glucose medium (Gibco BRL, USA) containing 10% FBS (Gibco BRL, USA) and an antibiotic-antimycotic agent (ThermoFisher Scientific, USA) at a seeding density of 5×10³ cells per cm². The plate was maintained at 5% CO₂ and 37° C. for 48 hours. The plate was then washed with PBS to remove nonattached cells, and the medium was replaced. The medium was replaced every 48 to 72 hours. When 70% confluency was achieved, 1×10⁶ cells were subcultured into T75 flasks (ThermoFisher Scientific, USA).

### Example 2 Isolation and Culture of Cochlear Explants

ICR mice 2 to 4 days after birth were purchased from DBL (Korea) and used. After disinfection with 70% ethanol, the head of the mouse was taken out using a blade, and the skull was cut in a sagittal plane. Cochlea was isolated after sequentially taking away the skin and temporal bone. The isolated cochlea was placed in a cold HEPES/HBSS solution (1×HBSS and 10 mM HEPES). After the cochlear jelly bone was taken away, the stria vascularis was taken away, and the organ of Corti including hair cells was separated from the spiral ganglion and obtained. At this time, the cochlea was divided into three parts from the center, that is, the apical turn, the middle turn, and the basal turn, and the organ of Corti was obtained from each of these three parts. Next, the tectorial membrane was taken away, and then the organ of Corti was carefully placed on a 9-mm-diameter plastic coverslip (SPL Life Sciences, Korea) with the basilar membrane facing down to obtain cochlear explants. The coverslip was placed in a 24-well culture dish, and 1 ml of explant culture medium (DEME/F12 medium containing 10% FBS, 1% N2 supplement, and 10 μg/ml ampicillin) was added into the well. The cochlear explants were transferred to an incubator at 5% CO₂ and 37° C. before drug treatment and co-culture with MSCs.

### Experimental Example 1 Confirmation of Ototoxic Hearing Loss Induction Using Cochlear Explants

It is known that ototoxic hearing loss is caused by excessive use of cisplatin, which is used as an anticancer drug. It is also known that damage to outer hair cells (OHC) and inner hair cells (IHC) of the cochlea leads to hearing loss. Hence, in order to determine whether ototoxic hearing loss is induced in cochlear explants, cochlear explants were treated with cisplatin at different concentrations, and the cell viability of OHCs and IHCs was examined through immunofluorescence analysis.

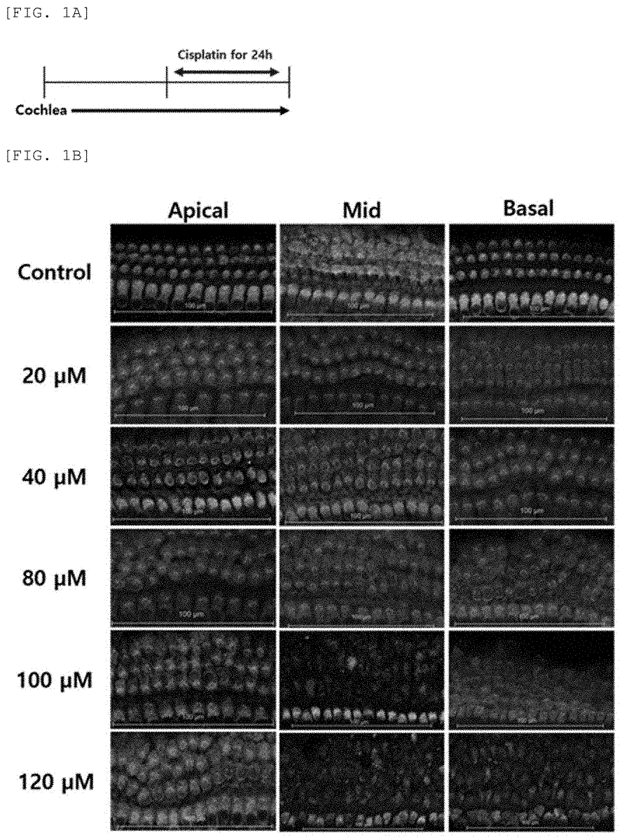

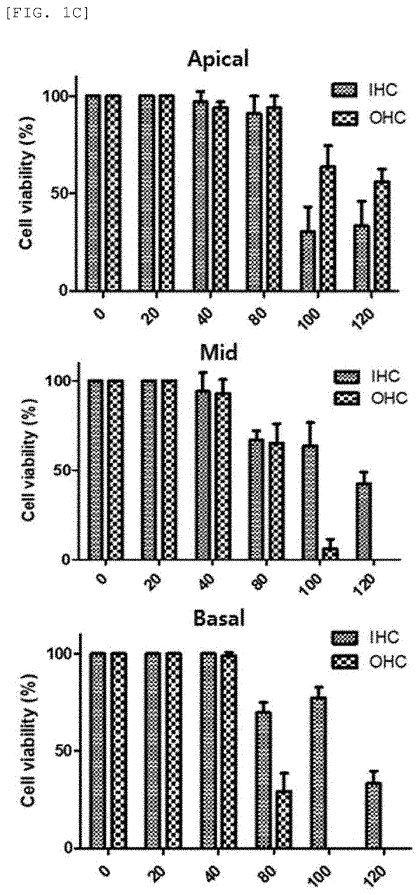

Specifically, the cochlear explants obtained in <Example 2> were treated with 20, 40, 80, 100, or 120 μM cisplatin (Sigma, USA) for 24 hours (FIG. 1A). After completion of the treatment, the cochlear explants were fixed with PBS containing 4% formalin for 15 minutes and washed with PBS three times. Next, after incubation with PBS containing 0.1% Triton X-100 for 10 minutes, blocking was performed with PBS containing 4% BSA for 30 minutes. Thereafter, incubation was performed for 1 hour with a rabbit anti-mouse myosin 7a primary antibody (1:400, abcam). After washing with PBS three times, incubation was performed for 1 hour with goat anti-rabbit IgG (H+L) Alexa Fluor 488 antibody (1:500, abcam) with Phalloidine Alexa Fluor 647 (1:1000, abcam). After washing with PBS, the coverslip was transferred to a slide and a drop of Fluoroshield with DAPI (Sigma, USA) was gently applied to the coverslip. The coverslip was sealed with clear nail polish and observed under a fluorescence microscope (FIG. 1B), and the number of OHCs and IHCs was measured to calculate the cell viability (FIG. 1C).

As a result, as illustrated in FIGS. 1A to 1C, since death of auditory hair cells increases depending on the concentration of cisplatin in cochlear explants, it has been confirmed that auditory hair cells are damaged by cisplatin and ototoxic hearing loss is induced. In particular, it has been confirmed that 100 μM cisplatin has the EC50 value of OHC in the apical turn but OHCs were almost killed in the middle turn and basal turn (FIG. 1C).

Therefore, as the cisplatin concentration for inducing ototoxic hearing loss in cochlear explants, 80 μM, which was a concentration having the EC50 value of OHC in the middle turn and basal turn as well, was selected, and the 80 μM cisplatin treatment group was used as a positive control.

### Experimental Example 2 Confirmation of MSC Using Fluorescence-Activated Cell Sorting

In order to investigate the effect of preventing or treating ototoxic hearing loss by MSC and substances derived therefrom, the properties of MSCs isolated from human bone marrow in <Example 1> and extracellular vesicles (EVs) derived therefrom were examined using FACS.

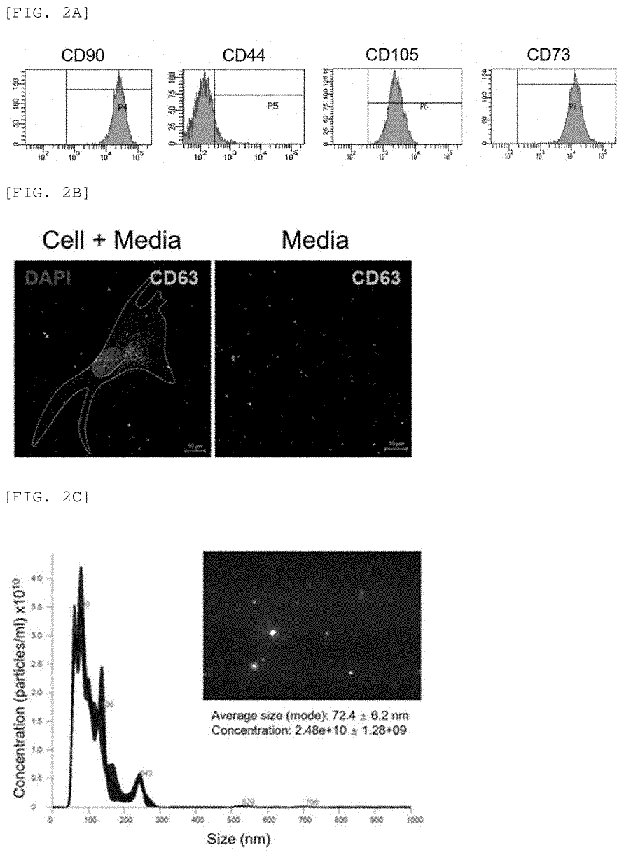

Specifically, the immune profile of MSCs isolated in <Example 1> was evaluated by fluorescence-activated cell sorting (FACS) using standards for MSCs as described in ISCT (International Society for Cellular Therapy) (REF). Cell surface markers were analyzed using a human MSC (hMSC) assay kit (BD sciences, USA). The hMSC Positive Cocktail (CD90 FITC, CD105 PerCP-Cy5.5 and CD73 APC) and PE hMSC Negative Cocktail (CD34, CD11b, CD19, CD45, HLA-DR) were used as positive and negative controls according to the manufacturer's procedure. The kit's hMSC Positive Isotype Control Cocktail (mIgG1κ FITC, mIgG1κ PerCP-Cy5.5 and mIgG1κ APC) and PE hMSC Negative Isotype Control Cocktail (mIgG1κ PE and mIgG2aκ PE) were also used as isotype controls. Samples were analyzed using a FACS Aria3 flow cytometer (Becton Dickinson, San Jose, CA, USA). Data were analyzed using FACS Diva software (FIG. 2A).

In order to observe EVs derived from MSCs isolated in <Example 1>, the MSCs isolated in <Example 1> and the culture medium in which the MSCs were cultured were each separated, and then immunofluorescence analysis was performed as described in <Experimental Example 1>. At this time, an exosome marker CD63 antibody was used as the primary antibody (FIG. 2B).

In order to determine the properties of EVs derived from MSCs contained in the culture medium, the culture medium was centrifuged to obtain a pellet and a supernatant. Thereafter, the size and concentration of EVs contained in the obtained pellet were measured using a nanoparticle tracking analyzer (NTA) (FIG. 2C).

As a result, as illustrated in FIGS. 2A to 2C, it has been confirmed that the MSCs isolated in <Example 1>showed CD90, CD105, CD73 and CD44 positive (FIG. 2A). CD63-positive exosomes have been identified in the MSCs and culture medium (FIG. 2B). It has been confirmed that MSC-derived EVs have a size of about 72.4±6.2 nm and a concentration of 2.48×10¹⁰±1.28×10⁹ (FIG. 2C).

It has been confirmed from the results that most of the EVs derived from MSCs isolated in <Example 1> were exosomes having a size of less than 100 nm.

### Experimental Example 3 Confirmation of Ototoxic Hearing Loss Preventing Effect in Cochlear Explants by MSC Pretreatment

In order to investigate the effect of MSCs to prevent or treat ototoxic hearing loss, after co-culture of MSCs and cochlear explants and treatment with cisplatin at the optimal concentration for inducing ototoxic hearing loss confirmed in <Experimental Example 1>, the cell viability of OHCs and IHCs was examined through immunofluorescence analysis.

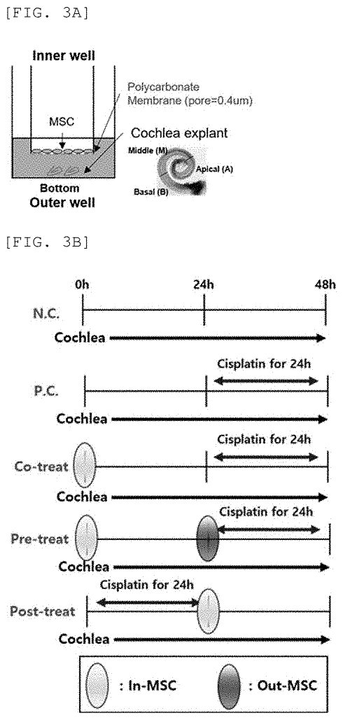

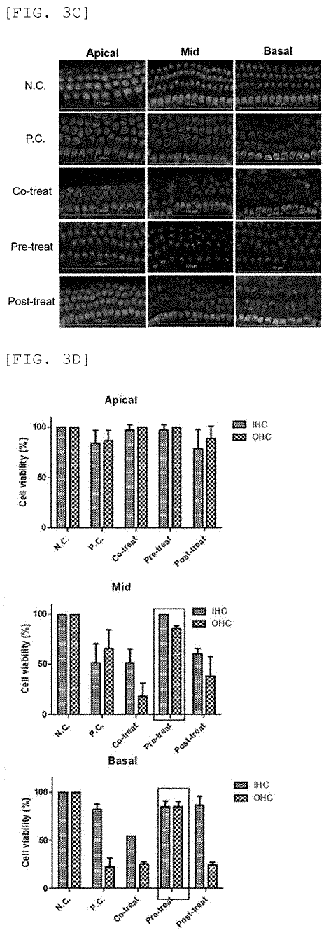

Specifically, as illustrated in the schematic diagram of FIG. 3A, MSCs isolated in <Example 1> were seeded in the inner well at a cell count of 1×10⁴ and treated with a MSC culture medium. The inner well included a polycarbonate membrane having a pore size of 0.4 μm to prevent cells from passing through. Next, co-culture was performed in a state where the cochlear explants cultured in <Example 2> were present in the outer well. As illustrated in the schematic diagram of FIG. 3B, experiments were performed by varying the time points of co-culture of cochlear explants and MSCs and treatment of cochlear explants with cisplatin. Specifically, in MSC co-treatment group (Co-treat), MSCs and cochlear explants were co-cultured 24 hours before the treatment of cochlear explants with cisplatin, and treatment with 80 μM cisplatin was performed for 24 hours while MSCs and cochlear explants were co-cultured. In MSC pre-treatment group (Pre-treat), MSCs and cochlear explants were co-cultured 24 hours before the treatment with cisplatin, and treatment with 80 μM cisplatin was performed for 24 hours in a state where the inner well containing MSCs was removed. In the MSC post-treatment group (Post-treat), cochlear explants were treated with 80 μM cisplatin for 24 hours and then co-cultured with MSCs for 24 hours.

After the experiment of each group was completed, the cell viability of OHCs and IHCs was examined through immunofluorescence analysis in the same manner as in <Experimental Example 1>.

As a result, as illustrated in FIGS. 3C and 3D, in the case of the co-treatment group, it has been confirmed that most OHCs in the middle turn and basal turn of the cochlear explants were killed after 48 hours. In particular, it has been confirmed that stereocilia disappeared from OHCs and IHCs survived. In the case of the post-treatment group as well, it has been confirmed that OHCs were damaged. On the other hand, in the pre-treatment group, OHCs survived in a row and stereocilia were also observed (FIG. 3C).

As a result of examining the number of cells, the viability of OHCs in the pre-treatment group was 86±2.14% in the middle turn and 84±5.4% in the basal turn (FIG. 3D). However, the cell viability in the co-treatment group and the post-treatment group was 18.2±13.2% and 38.4±19.5% in the middle turn, respectively. The cell viability was 25.3±2.6% in the co-treatment group and 24.2±3% in the post-treatment group (FIG. 3D).

It can be seen from the results that MSCs co-cultured with cochlear explants are effective in preventing damage to IHCs and OHCs caused by cisplatin. On the other hand, it can be seen that OHCs already damaged by cisplatin are rarely affected by MSCs.

### Experimental Example 4 Confirmation of Ototoxic Hearing Loss Preventing Effect in Cochlear Explants Depending on MSC Pretreatment Time

In order to investigate the effect of preventing ototoxic hearing loss in cochlear explants depending on the MSC pre-treatment time, MSCs and cochlear explants were co-cultured for different times, MSCs were removed, then the cochlear explants were treated with cisplatin, and the cell viability of OHCs and IHCs was examined through immunofluorescence analysis.

Specifically, a pre-treatment group (Pre-treat) was prepared in the same manner as in <Experimental Example 3> except that the time for co-culture of MSCs and cochlear explants was varied to 2, 12, 18, 24 and 48 hours. After the experiment of each group was completed, the cell viability of OHCs and IHCs was examined through immunofluorescence analysis in the same manner as in <Experimental Example 1>.

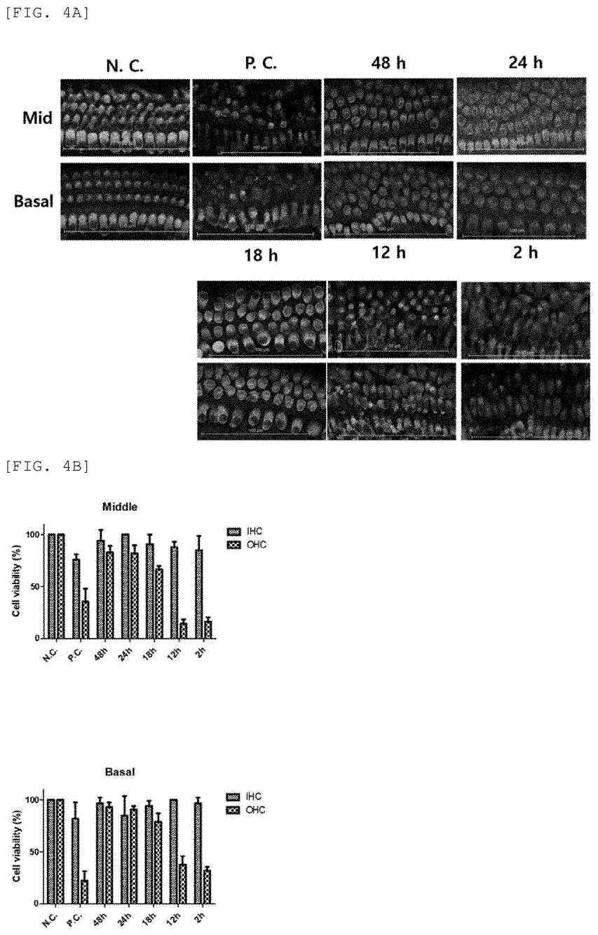

As a result, as illustrated in FIGS. 4A and 4B, it has been confirmed that OHCs were killed in the 12-hour pre-treatment group. Meanwhile, the cell viability of OHCs gradually increased in the 18 hours or more pre-treatment groups. At this time, the cell viability in the middle turn was 66.7±3% (18-hour pre-treatment group), 81.8±8% (24-hour pre-treatment group), and 82.8±6.3% (48-hour pre-treatment group), respectively. The cell viability of OHCs in the basal turn was higher than that in the middle turn. At this time, the cell viability in the basal turn was 78.8±8% (18-hour pre-treatment group), 91±3% (24-hour pre-treatment group), and 93±9% (48-hour pre-treatment group), respectively. In particular, it has been confirmed that the ratio of OHCs damaged by cisplatin decreases depending on the time for exposure to MSCs (FIGS. 4A and 4B).

It can be seen from the results that MSCs co-cultured with cochlear explants for 18 hours or more are far effective in preventing damage to IHCs and OHCs caused by cisplatin.

### Experimental Example 5 Confirmation of Ototoxic Hearing Loss Preventing Effect in Cochlear Explants by MSC-Derived Exosome Pretreatment

In order to investigate the effect of exosomes derived from MSCs to prevent ototoxic hearing loss in cochlear explants, MSCs and cochlear explants were co-cultured for different times, MSCs were removed, then the cochlear explants were treated with cisplatin, and the cell viability of OHCs and IHCs was examined through immunofluorescence analysis.

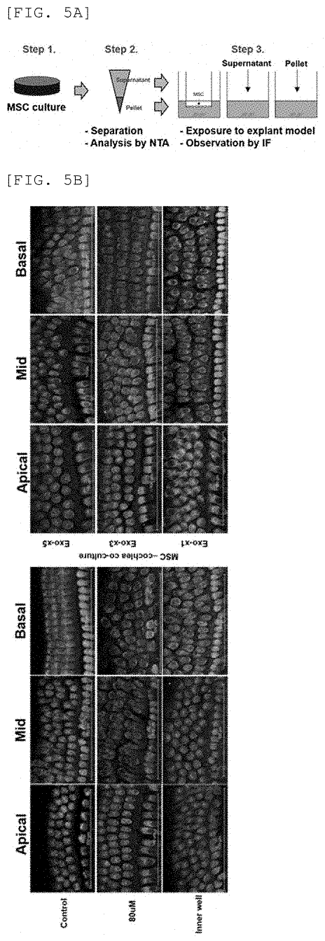

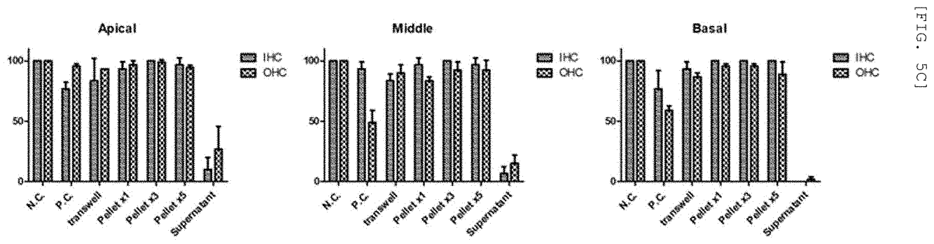

Specifically, as illustrated in the schematic diagram of FIG. 5A, the exosomes isolated and identified in <Example 2> were prepared to have a 10-fold concentration. Next, in order to compare with the MSC pre-treatment group in <Experimental Example 3>, the exosomes were diluted by ×1, ×3, and ×5, then the cochlear explants cultured in <Example 2> were treated with the exosomes for 24 hours. After completion of the treatment with exosomes, treatment with 80 μM cisplatin was performed for 24 hours. In order to compare with the group not containing MSC-derived exosomes, the supernatant obtained in <Example 2> was treated in the same manner as above. After completion of the treatment, treatment with 80 μM cisplatin was performed for 24 hours.

After the experiment of each group was completed, the cell viability of OHCs and IHCs was examined through immunofluorescence analysis in the same manner as in <Experimental Example 1>.

As a result, as illustrated in FIGS. 5B and 5C, it has been confirmed that significant death of IHCs and OHCs was observed in all of the apical turn, middle turn and basal turn in the supernatant pre-treatment group (Supernatant) that does not contain exosomes but the cell viability of IHCs and OHCs was excellent in all of the apical turn, middle turn and basal turn in the exosome pre-treatment group (FIGS. 5B and 5C).

It can be seen from the results that MSC-derived exosomes are effective in preventing damage to IHCs and OHCs caused by cisplatin.

### Experimental Example 6 Confirmation of HSP70 Protein Expression in MSCs Co-Cultured with Cochlear Explants and Exosomes Derived Therefrom

HSP70 is known to have an effect of inhibiting damage to auditory hair cells (Y Takada et al. 2015). Through <Experimental Example 3> and <Experimental Example 4>, the effect of preventing damage to IHCs and OHCs caused by cisplatin has been confirmed in the group where cochlear explants are pretreated with MSC. Therefore, in order to examine the ototoxic hearing loss preventing effect of MSCs co-cultured with cochlear explants and exosomes derived therefrom, the HSP70 protein expression in these MSCs and exosomes was examined by Western blot analysis.

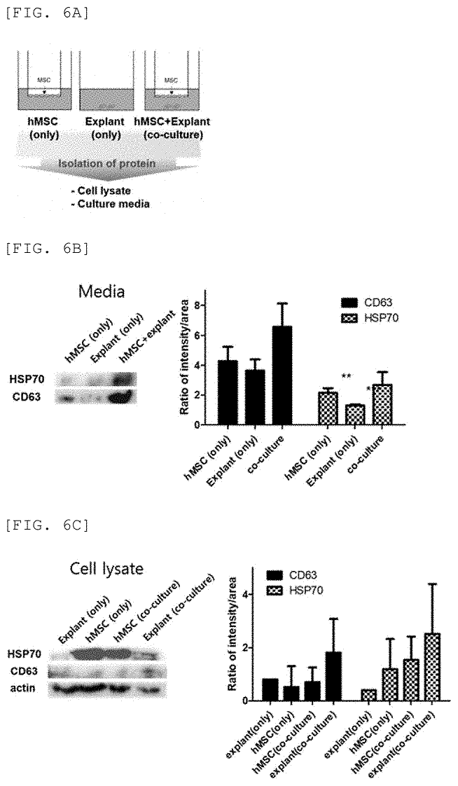

Specifically, as illustrated in the schematic diagram of FIG. 6A, MSCs obtained in <Example 1> and cochlear explants obtained in <Example 2> were co-cultured for 24 hours in the same manner as in <Experimental Example 3>, and then centrifuged to obtain cells and a culture solution. Subsequently, in order to obtain exosome proteins in the cells and culture solution, the cells and culture solution were reacted for 15 minutes using a lysis buffer, and then centrifuged to obtain cell lysates. The concentration of each sample was quantified to 60 μg through Bradford assay. Subsequently, the sample was separated by electrophoresis on a 12% acrylamide gel and transferred to a PVDF membrane. The membrane was reacted with each of an exosome marker CD63 antibody and an HSP70 antibody as primary antibodies at 4° C. overnight, and reacted with HRP-conjugated IgG antibody as a secondary antibody for 1 hour at room temperature. Then, immunoreactive bands were detected and graphed using ChemiDOC. As a control group, a group in which only MSCs were cultured without cochlear explants and a group in which only cochlear explants were cultured without MSCs were used.

As a result, as illustrated in FIGS. 6B and 6C, it has been confirmed that the CD63 and HSP70 protein expression is higher in the culture solution of the group [hMSC (only)] in which only MSCs are cultured than in the culture solution of the group [Explant (only)] in which only cochlear explants are cultured. It has been confirmed that the CD63 and HSP70 protein expression is far higher in the culture solution of the group [co-culture] in which MSCs and cochlear explants are co-cultured (FIG. 6B).

It can be seen from the results that a large amount of HSP70 protein is contained in MSC-derived exosomes and exosomes derived from MSCs co-cultured with cochlear explants, this inhibits damage to IHCs and OHCs, and a hearing loss preventing effect is thus exerted. It can be seen that the co-culture solution of cochlear explants and MSCs contains a more amount of exosomes containing HSP70 protein, and thus the hearing loss preventing effect is superior.

It has been confirmed that the CD63 and HSP70 protein expression is higher in MSCs [hMSC (co-culture)] of the group in which MSCs and cochlear explants are co-cultured than in MSCs [hMSC (only)] of the group in which only MSCs are cultured. It has been confirmed that the CD63 and HSP70 protein expression is far higher in cochlear explants [explant (co-culture)] of the group in which MSCs and cochlear explants are co-cultured.

It can be seen from the results that HSP70 protein and exosomes containing the protein increase in MSCs co-cultured with cochlear explants, this enhances the effect of inhibiting damage to IHCs and OHCs, and an excellent hearing loss preventing effect is thus exerted. It can be seen that HSP70 protein and exosomes containing the protein increase in cochlear explants co-cultured with MSCs as well as in MSCs, this inhibits damage to IHCs and OHCs, and an excellent hearing loss preventing effect is thus exerted.

### INDUSTRIAL APPLICABILITY

Mesenchymal stem cells co-cultured with cochlear explants according to the present invention, exosomes contained in a co-culture solution of mesenchymal stem cells and cochlear explants, and exosomes contained in a mesenchymal stem cell culture solution of the present invention are effective in preventing damage to the inner hair cells and outer hair cells of the cochlea caused by ototoxic drugs since a large amount of HSP70 protein is contained therein, and thus these can be usefully utilized as an active ingredient of a composition for preventing hearing loss.

1. A method for preventing hearing loss, the method comprising administering a pharmaceutically effective amount of mesenchymal stem cells (MSC) co-cultured with cochlear explants, a co-culture solution of mesenchymal stem cells and cochlear explants exosomes isolated from the co-culture solution, a mesenchymal stem cell culture solution or exosomes isolated from the culture solution to a subject.

2. The method according to claim 1, wherein the mesenchymal stem cells and the cochlear explants are co-cultured in a state of being spatially separated from each other in a co-culture medium.

3. The method according to claim 2, wherein the mesenchymal stem cells and the cochlear explants are co-cultured in a state of being spatially separated from each other in a co-culture medium as the mesenchymal stem cells are positioned in a transwell upper chamber and the cochlear explants are positioned in a transwell lower chamber.

4. The method according to claim 1, wherein the co-culture is performed for 12 hours or more.

5. The method according to claim 1, wherein the mesenchymal stem cells are mesenchymal stem cells derived from bone marrow, fat, umbilical cord, umbilical cord blood or tonsil.

6. The method according to claim 1, wherein expression of HSP70 protein increases in the mesenchymal stem cells co-cultured with cochlear explants and the co-culture solution of mesenchymal stem cells and cochlear explants the exosomes isolated from the co-culture solution, the mesenchymal stem cell culture solution and the exosomes isolated from the culture solution contain HSP70 protein.

7. The method according to claim 1, wherein the mesenchymal stem cells co-cultured with cochlear explants, the co-culture solution of mesenchymal stem cells and cochlear explants, the exosomes isolated from the co-culture solution, the mesenchymal stem cell culture solution and the exosomes isolated from the culture solution protect inner hair cells and outer hair cells of cochlea from damage.

8. The method according to claim 1, wherein the exosomes have a diameter of 40 to 180 nm.

9. The method according to claim 1, wherein the hearing loss is sensorineural hearing loss.

10. The method according to claim 9, wherein the sensorineural hearing loss is any one or more selected from the group consisting of ototoxic hearing loss, organ of Corti damage-induced hearing loss due to viral infection, chronic otitis media-induced hearing loss, age-related hearing loss, noise-induced hearing loss, sudden hearing loss, autoimmune hearing loss, vascular ischemic hearing loss, head injury hearing loss, and hereditary hearing loss.

11. The method according to claim 10, wherein the ototoxic hearing loss is caused by an ototoxic drug.

12. The method according to claim 11, wherein the ototoxic drug is any one or more selected from the group consisting of cisplatin, carboplatin, amikacin, arbekacin, kanamycin, gentamicin, neomycin, netilmicin, dibekacin, sisomycin, streptomycin, tobramycin, livodomycin, paromomycin, acetazolamide, furosemide, bumetanide and ethacrynic acid.

13. The method according to claim 9, wherein the sensorineural hearing loss is caused by damage to inner hair cells of cochlea, outer hair cells of cochlea or surrounding tissues.

14. The method according to claim 1, further administering cochlear explants co-cultered with mesenchymal stem cells.

15.-20. (canceled)

21. A method for preparing mesenchymal stem cells for preventing hearing loss comprising the following step 1) and 2), or preparing mesenchymal stem cells and cochlear explants for preventing hearing loss comprising the following step 1):
1) co-culturing cochlear explants and mesenchymal stem cells; and
2) obtaining co-cultured mesenchymal stem cells.

22. The method according to claim 15, wherein the mesenchymal stem cells and the cochlear explants are co-cultured in a state of being spatially separated from each other in a co-culture medium as the mesenchymal stem cells are positioned in a transwell upper chamber and the cochlear explants are positioned in a transwell lower chamber in step 1).

23. (canceled)

24. A method for preparing a co-culture solution of mesenchymal stem cells and cochlear explants for preventing hearing loss comprising the following step 1) and 2), or preparing exosomes isolated from the co-culture solution of mesenchymal stem cells and cochlear explants for preventing hearing loss comprising the following step 1) to 3):
1) co-culturing mesenchymal stem cells and cochlear explants in a co-culture medium;
2) recovering a cell culture supernatant of co-cultured mesenchymal stem cells and cochlear explants; and
3) isolating exosomes from the recovered cell culture supernatant and purifying the exosomes.

25. The method according to claim 17, wherein the mesenchymal stem cells and the cochlear explants are co-cultured in a state of being spatially separated from each other in a co-culture medium as the mesenchymal stem cells are positioned in a transwell upper chamber and the cochlear explants are positioned in a transwell lower chamber in step 1).

26. The method according to claim 17, wherein the co-culture in step 1) is performed for 12 hours or more.

27.-31. (canceled)
